(19) United States
(12) Patent Application Publication
(10) Pub. No.: US 2005/0285043 A1
(43) Pub. Date: Dec. 29, 2005

(54) X-ray detector array for both imgaging and measuring dose

(76) Inventors: Augusto Nascetti, Aachen (DE); Martin J. Powell, Horley (GB); Anthony R. Franklin, East Grinstead (GB)

Correspondence Address: PHILIPS INTELLECTUAL PROPERTY & STANDARDS, P.O. BOX 3001, BRIARCLIFF MANOR, NY, 10510, US

(21) Appl. No.: 10/515,466

(22) PCT Filed: May 15, 2003
(86) PCT No.: PCT/IB03/02065

§ 371 Date: Nov. 22, 2004

(30) Foreign Application Priority Data
May 24, 2002 (GB) 0212001.2

(51) Int. Cl. G01J 1/00

(52) U.S. Cl. 250/370.09

(57) ABSTRACT

An X-ray detector apparatus has an array of pixels arranged into a plurality of sub-arrays (40). The pixels in each sub-array (40) share a common output (42). The detector is operable in two modes, a dose sensing mode in which a switching arrangement (50) is turned off and charge flow in response to incident radiation is partially coupled through an off-capacitance of the switching arrangement (50) to the output, and a read out mode in which the switching arrangement is turned on to allow charge to flow between the charge storage element and the output (42) for measurement as a detection signal. The switching arrangement (50) is turned on by first and second control signals to enable a single pixel within the sub-array (40) to be selected. Thus, the resolution of normal read out is per-pixel whereas the resolution of dose sensing is per-sub-array.

The invention relates to an X-ray detector and to an X-ray examination apparatus, which uses the detector. In particular, the detector is for providing image signals as well as exposure control signals by having exposure measurement circuitry integrated with solid state X-ray detector circuitry. This enables real time control of the X-ray exposure during an image acquisition process.

It is well known that the X-ray exposure of a patient should be controlled as a function of the absorptivity of the tissue under examination. For example, overexposed areas of high brightness may occur in the image, for example is caused by X-rays which are not (or only hardly) attenuated by the object to be examined, for example a patient. Tissue having a low X-ray absorptivity, for example lung tissue, will provide less attenuation and therefore requires less X-ray exposure to obtain an image of given contrast and to prevent saturation of the image detector.

Configurations of known X-ray examination apparatus are well known to those skilled in the art. Typically, the apparatus includes an X-ray source for irradiating a patient to be radiologically examined, by means of an X-ray beam. Due to local differences in the X-ray absorptivity within the patient, an X-ray image is formed. The X-ray detector derives an image signal from the X-ray image. In a detector using an optical sensor, the detector has a conversion layer or surface for converting the incident X-ray energy into optical signals. In the past, these optical signals have largely been detected by an image intensifier pick-up chain, which includes an X-ray image intensifier and a television camera.

A known X-ray examination apparatus of this type is disclosed in U.S. Pat. No. 5,461,658. This document additionally discloses an exposure control system in which an auxiliary light detection system utilizes local brightness values in the optical image in order to adjust the X-ray source. This auxiliary light detection system includes a CCD sensor for locally measuring the brightness in the optical image. The exposure control system derives a control signal from the measured brightness values, the control signal being used to adjust the X-ray apparatus in such a manner that an X-ray image of high diagnostic quality is formed and displayed, namely such that small details are included in the X-ray image and suitably visibly reproduced. The control signal controls the intensity and/or the energy of the X-ray beam and can also be used to control the amplification of the image signal. Both steps influence the signal level of the image signal directly or indirectly.

More recently, the use of a solid state X-ray detectors have been proposed. There are two basic configurations for such devices.

In a so-called “indirect” detector arrangement, the incident X-ray radiation is first converted into light. An array of photosensitive cells is provided, each comprising a light-sensitive element (photodiode), and a charge storage device (which may be a separate element or it may be the self-capacitance of the photodiode).

In a so-called “direct” detector arrangement, an X-ray sensitive photoconductor is used to convert the X-rays directly into electrons. Since the photoconductor has no self-capacitance, a capacitor is fabricated by thin film techniques to act as a charge storage device.

During X-ray exposure, the light incident on each cell is stored as a level of charge on the charge storage device, to be read out at the end of the exposure period. The read out of charges stored effectively resets the image sensor, so this can only be carried out at the end of the X-ray exposure period. Thus, it is not possible to use the output signals from an image sensor of this type to control the exposure period in real time, because such outputs are only available at the end of exposure. The nature of the solid state image sensor device also prevents the type of feedback control described above using CCDs to be implemented.

One possible way to achieve dose control is to analyse the obtained image, and then to repeat the image acquisition process with a different exposure level. Of course, this increases the overall exposure of the patient to potentially harmful X-ray radiation, and is also not appropriate for rapidly changing images, or where images from different viewpoints are required in rapid succession.

External dose sensing arrangements have been proposed which are independent of the solid state image detector, but these can degrade the image quality. There is therefore a need for a dose sensing arrangement which enables real time dose control and which can be used with solid state image sensors.

It has also been proposed to combine dose sensing elements into the normal image sensing pixel layout. Typically, a pixel design with integrated dose sensing elements requires separate read out lines for the dose sensing signal and the image read out signal, and separate read out amplifiers for the two types of signal. Typically, each column of pixels has an allocated read out line and amplifier, and additional amplifiers are provided for the dose sensing function.

An example of integrated dose sensing is in WO 02/25314 A1.

According to the invention, there is provided an X-ray detector apparatus comprising an array of detector pixels, each pixel comprising a conversion element for converting incident radiation into a charge flow, a charge storage element and a switching arrangement enabling the charge stored to be provided to an output of the pixel, wherein the array of pixels is arranged into a plurality of sub-arrays, each sub array comprising a plurality of pixels, the pixels in each sub-array sharing a common output, and wherein the detector apparatus is operable in two modes, a first mode in which the switching arrangement is turned off and charge flow in response to incident radiation is partially coupled through an off-capacitance of the switching arrangement to the output for measurement as a dose sensing signal, and a second mode in which the switching arrangement is turned on to allow charge to flow between the charge storage element and the output for measurement as a detection signal, and wherein the switching arrangement is turned on by first and second control signals which enable a single pixel within the sub-array to be selected.

In this arrangement, pixels are divided into sub-arrays which share a common output. This common output can be used for dose sensing during exposure, and the dose sensing is performed with a resolution corresponding to the size of the sub-arrays. The number of read out amplifiers is reduced to one per sub-array of pixels, and this is achieved by having multiplexing in the pixels. In particular, the switching arrangement in each pixel is responsive to two control signals so that a single pixel within the sub-array can be selected. The same common output can thus be used for measurement of an individual pixel signal, so that the resolution of the detector is not reduced. The switching arrangement enables the same output to be used for dose sensing and conventional read out by providing capacitive coupling to the read out line when the switching arrangement is turned off, and providing direct conductive coupling when the switching arrangement is turned on.

This detector is preferably used in an X-ray examination apparatus comprising an X-ray source for exposing an object to be examined to X-ray energy. The detector receives an X-ray image after attenuation by the object to be examined.

The apparatus may further comprise a phosphor conversion layer for converting an incident X-ray signal into an optical signal, and the conversion element then comprises an optical sensor, such as a photodiode. The charge storage element may then be a separate element in parallel with the photodiode, or it may comprise the self-capacitance of the photodiode.

Alternatively, the conversion element may comprise a photoconductor and a capacitor, which converts the X-ray radiation directly into an electron charge flow.

The switching arrangement may comprise first and second thin film transistors in series between the conversion element and the output, one of the transistors being gated by a row select control signal and the other of the transistors being gated by a column select control signal. In this way, two transistors provide an “AND” function so that an individual pixel within a two dimensional sub-array may be selected. This enables an individual pixel to be recharged by charge flow along the output.

Alternatively, the switching arrangement may comprise a first thin film transistor in series between the conversion element and the output and a second thin film transistor, wherein the second thin film transistor is gated by a first control signal for switching a second control signal to the gate of the first transistor. In this arrangement, the second transistor provides the “AND” function, with one of the control signals on the source/drain and the other on the gate. When the second transistor is turned off (during X-ray exposure), the gate of the first transistor forms a floating node, which increases the source-drain capacitance of the first transistor.

Each pixel may further comprise an additional capacitor between the gate of the first transistor and the conversion element. This enables the dose sensing signal to be matched to the read-out signal.

The pixels are preferably arranged in rows and columns, wherein each sub-array comprises a plurality of rows and columns.

A plurality of first control lines for carrying the first control signals can then be provided, the number of first control lines corresponding to the number of rows in each sub-array with each first control line being provided to one row of each sub-array, and a plurality of second control lines for carrying the second control signals can be provided, the number of second control lines corresponding to the number of columns in each sub-array with each second control line being provided to one column of each sub-array.

In this way, the control signals for each sub-array of pixels are shared, so that each pixel sub-array can be read out simultaneously. This reduces the number of control lines needed to interface with the device. A read out amplifier is provided only for each sub-array of pixels, and the multiplexing within the pixel layout reduces the number of amplifiers needed whilst avoiding the need for additional multiplexing circuitry.

Examples of the invention will now be described in detail with reference to the accompanying drawings, in which:

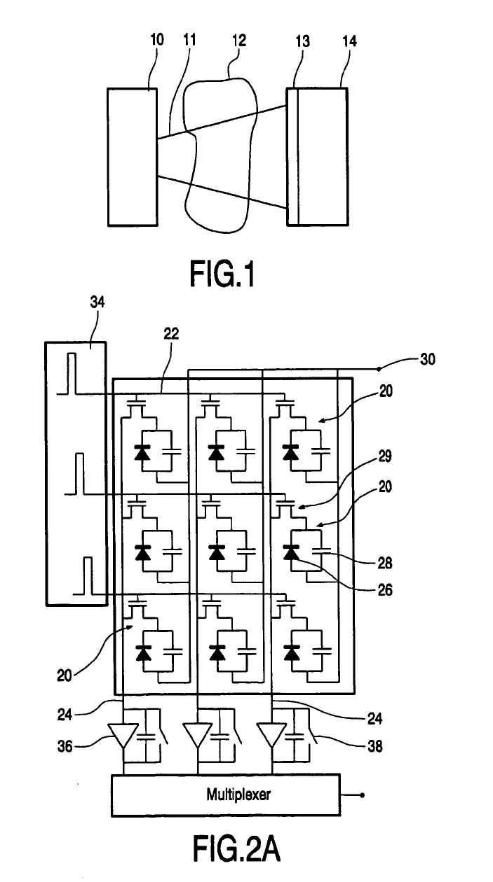

FIG. 1 shows a known X-ray examination apparatus;

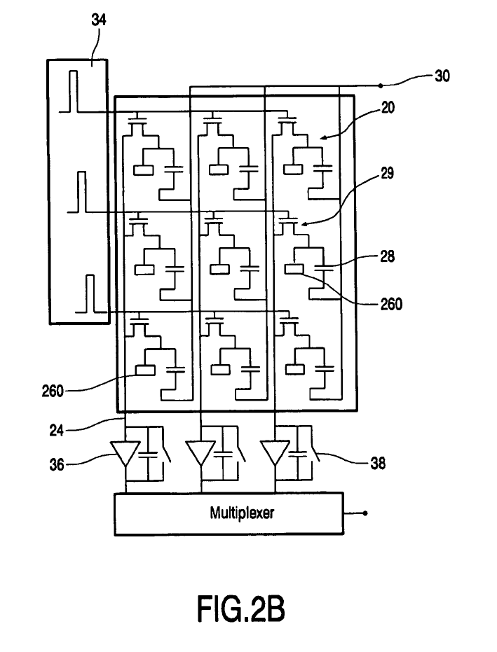

FIG. 2A shows a first known pixel layout for a solid state image sensor used in the apparatus of FIG. 1;

FIG. 2B shows a second known pixel layout for a solid state image sensor used in the apparatus of FIG. 1;

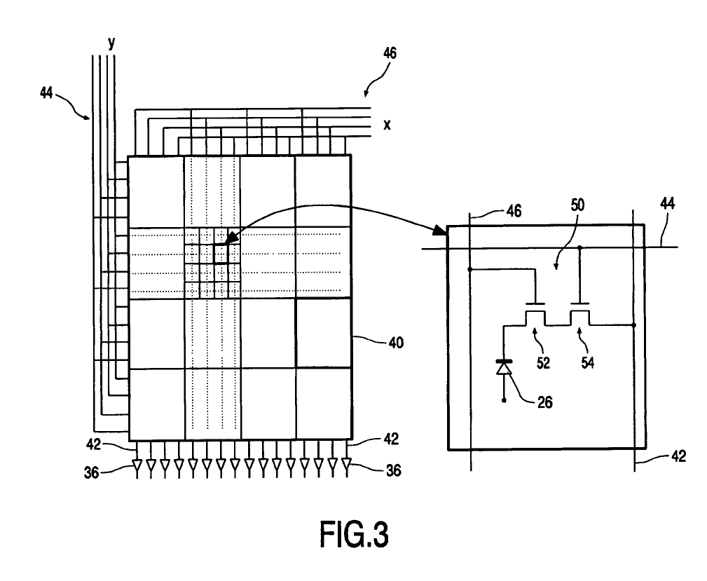

FIG. 3 shows a first modified pixel arrangement according to the invention;

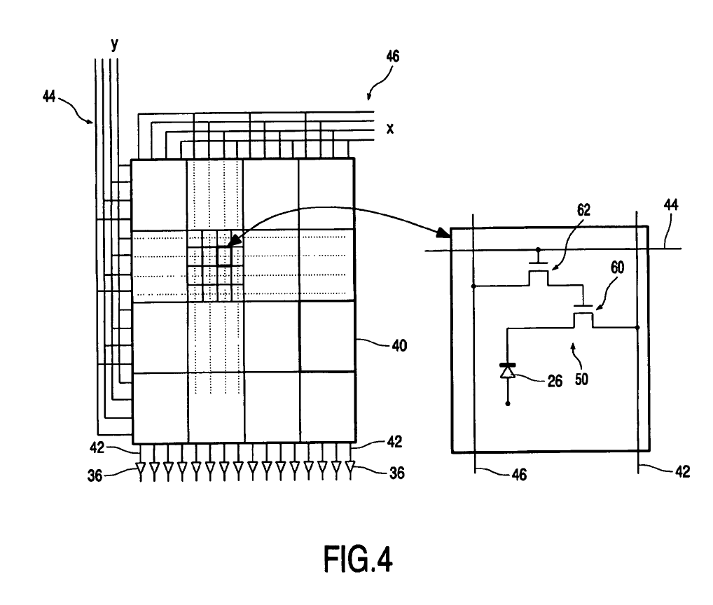

FIG. 4 shows a second modified pixel arrangement according to the invention;

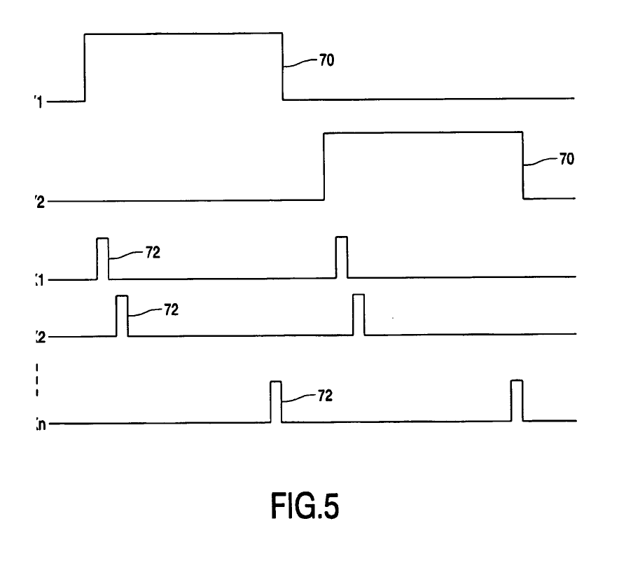

FIG. 5 is a timing diagram for explaining further the operation of the pixel arrangement of FIG. 4;

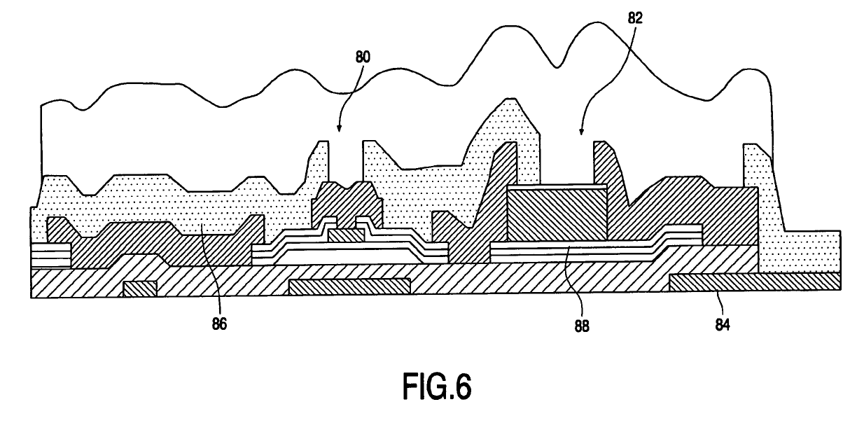

FIGS. 6 to 9 show different fabrication technologies which may be applied to the pixel arrangement of the invention;

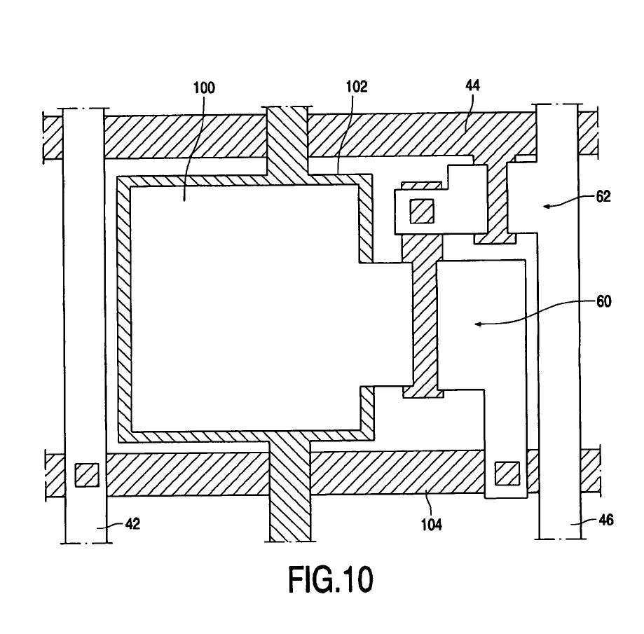

FIGS. 10 to 12 show in more detail how the pixel arrangement of FIG. 4 may be implemented using different technologies; and

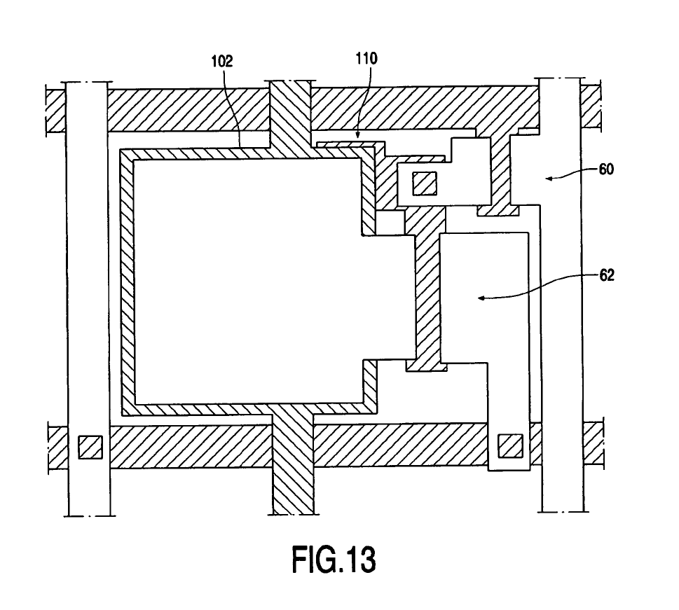

FIGS. 13 to 15 show modifications to the implementations of FIGS. 10 to 12.

FIG. 1 shows a known X-ray examination apparatus which includes an X-ray source 10 for irradiating an object 12 to be examined, for example a patient to be radiologically examined, by means of an X-ray beam 11. Due to local differences in the X-ray absorption within the patient, an X-ray image is formed on an X-ray-sensitive surface 13 of the X-ray detector 14.

One known design of X-ray detector 14 uses a solid state optical image sensor. The incident X-ray radiation is converted into light using a phosphor scintillator 13. This light can be detected by the solid-state device 14. Alternatively, an X-ray sensitive phootoconductor can be used to convert the X-rays directly into electrons.

FIG. 2A shows one known design for the solid state optical image sensor. The sensor comprises an array of pixels 20 arranged in rows and columns. Rows of pixels share a row address line 22, and columns of pixels share a readout line 24. Each pixel comprises a photodiode 26 in parallel with a charge storage capacitor 28. This capacitor 28 may be a separate component, or else it may simply comprise the self-capacitance of the photodiode 26. This parallel combination is connected in series with a thin film transistor 29 between a common electrode 30 and the column readout line 24 for that particular pixel. The pixel array is provided on a glass substrate 32. Row driver circuitry 34 provides signals for the row address lines 22, and the column readout lines 24 provide an output from the substrate 32, and each column readout line 24 is associated with a respective charge sensitive amplifier 36.

The function of the photodiode is to convert the incident radiation into a flow of charge which alters the level of charge stored on the capacitor. In the case of direct conversion of the radiation using a photoconductor, the capacitor 28 is implemented as a separate thin film component, and again the level of charge stored is a function of the flow of charge from the photoconductor. FIG. 2B shows a known design of solid state direct X-ray detector. The same references are used as in FIG. 2A for the same components. The photoconductor 260 is biased to a suitable operating voltage. The photoconductor and capacitor effectively replaces the phosphor conversion layer and photodiode in the arrangement of FIG. 2A.

In operation of the image sensor device, the capacitors 28 are all charged to an initial value. This is achieved by the previous image acquisition or else may be achieved with an initial reset pulse on all row conductors 22. The charge sensitive amplifiers are reset using reset switches 38.

During X-ray exposure, light incident on the photodiodes 26 causes charge to flow in the reverse-bias direction through the photodiodes. This current is sourced by the capacitors 28 and results in a drop in the voltage level on those capacitors. Alternatively, the charge flow through the photoconductor 260 drains the charge from the capacitors 28.

At the end of X-ray exposure, row pulses are applied to each row conductor 22 in turn in order to switch on the transistors 29 of the pixels in that row. The capacitors 28 are then recharged to the initial voltage by currents flowing between the common electrode 30 and the column readout lines 24 and through the transistor switches. In the example shown, these currents will be sourced by the charge sensitive amplifiers 36, rather than flow to them. The amount of charge required to recharge the capacitors 28 to the original level is an indication of the amount of discharge of the storage capacitor 28, which in turn is an indication of the exposure of the pixel to incident radiation. This flow of charge is measured by the charge sensitive amplifiers. This procedure is repeated for each row to enable a full image to be recovered.

A problem with the use of solid-state image sensors of this type is that a pixel signal is only obtained during the read out stage, after the exposure has been completed. As will be apparent from the above description, any read out of signals results in recharging of the pixel capacitors 28, and effectively resets those pixels. Therefore, it is not possible to take samples during the image acquisition process, and the image sensor design does not therefore allow real-time exposure measurements to be obtained.

In accordance with the invention, the pixels are designed to enable a dose sensing function to be performed, as well as providing a multiplexing function which enables a reduction in the number of read out amplifiers required.

In the following description, optical detector pixels are shown with modification to provide the dose sensing function of the invention. However, the invention applies equally to direct detection schemes such as shown in FIG. 2B.

FIG. 3 shows a first pixel of the invention. Throughout the Figures, the same reference numbers will be used for the same components, and description of those components will not be repeated.

As shown in FIG. 3, the detector has an array of detector pixels which is arranged into a plurality of sub-arrays 40. Each sub-array 40 comprises a plurality of pixels also arranged in rows and columns. The pixels in each sub-array share a common output 42, and there is one read-out amplifier 36 associated with each common output. During read out of the device, one pixel from each sub-array is read out simultaneously. In order to select an individual pixel from each sub array 40, each pixel is associated with a row control line 44 and a column control line 46. The row control lines 44 form a set of control lines which are shared between the different sub-arrays 40, and similarly the column control lines 46 form a set of control lines which are shared between the different sub-arrays 40. 10. The number of control lines in set 44 corresponds to the number of rows in each sub-array and the number of control lines in set 46 corresponds to the number of columns in each sub-array.

FIG. 3 shows one pixel in enlarged form. As for the convention pixel configuration, each pixel has a conversion element 26 for converting incident radiation into a charge flow, a charge storage element which may be the intrinsic self-capacitance, and a switching arrangement 50 enabling the charge stored to be provided to the output 42 of the pixel. The conversion element is shown in the following drawings as an optical photodiode, but it will be appreciated that the invention is equally applicable to direct conversion elements.

In accordance with the invention, the switching arrangement 50 is able to select an individual pixel within a sub-array 40 by using two control signals, namely the signals on the row and column control lines 44,46.

In the example of FIG. 3, the switching arrangement 50 comprises first and second thin film transistors 52, 54 in series between the conversion element and the output 42. The first transistor 52 is gated by a column select control signal on the column control line 46, and the second transistor 54 is gated by a row select control signal on the row control line 44. In this way, the two transistors 52, 54 provide an “AND” function so that an individual pixel within the two dimensional sub-array 40 may be selected. During read out, an individual pixel is recharged by charge flow between the output 42 and the photodiode 26, so that the resolution of the read out is per-pixel.

The pixel configuration of the invention also enables a dose sensing output to be provided during exposure. Thus, the detector is operable in two modes. In a first mode, which is the exposure mode, the switching arrangement 50 is turned off and charge flow in response to incident radiation is partially coupled through the source-drain capacitance of the two transistors 52, 54, which are both turned off. The way in which this capacitive coupling can provide a dose sensing signal which does not destroy the read out signal will now be described.

In conventional manner, the voltage on the pixel capacitor 28 is preset to a known level before the image acquisition process. During X-ray exposure, the photodiode 26 provides a flow of charge which is proportional to the dose incident on the pixel. Part of this charge is stored on the pixel capacitor, while the other part flows on to the off-capacitance of the switching arrangement 50. This causes a corresponding flow of charge along the read out line 42. The charge sensitive amplifier 36 measures this flow of charge. All pixels in a sub-array 40 are associated with the signal read out line 42, so that the charge flow is summed for all pixels in the sub-array, and the resolution of the dose sensing signal is per sub-array rather than per pixel. The charge sensitive amplifier 46 maintains a fixed potential at its input, so that cross talk from one pixel cell to another does not arise.

At the end of the X-ray exposure, the pixels are read out in conventional way by switching on the switching arrangement to allow a charge to flow along the readout line 42 which recharges the pixel capacitor 28. The is the second mode of operation. However, charge also flows to the off-capacitance of the switching arrangement 50, so that charges flowing to or from this off-capacitance during X-ray exposure are not lost, but are recovered when the image read out process takes place.

The off-capacitance is significantly smaller than the pixel capacitor, so that the dose sensing signal (which is effectively a charge leakage across the turned off transistors) is relatively small. The transistor designs will be selected to provide appropriate levels of this capacitance. The summing of these signals for a group of pixels assists in measurement of the charge flow, but enables only a small increase in switching noise during pixel read out.

The pixel configuration of the invention enables the number of read out amplifiers to be reduced to one per sub-array of pixels, and this is achieved by having multiplexing in the pixels. The same common output is used for read out of individual pixel signals as for dose sensing of a sub-array of pixels, so that the resolution of the detector is not reduced. The switching arrangement enables the same output to be used for dose sensing and conventional read out by providing capacitive coupling to the read out line when the switching arrangement is turned off, and providing direct conductive coupling when the switching arrangement is turned on.

FIG. 4 shows an alternative pixel layout. The operation is the same as for the example of FIG. 3, but the switching arrangement 50 has a different design. The switching arrangement 50 has a first thin film transistor 60 in series between the photodiode 26 and the output 42 and a second thin film transistor 62. The second thin film transistor 62 is gated by the row select control signal from the row control signal line 44 and switches the column select control signal from the column control signal line 46 to the gate of the first transistor 60. In this way, the second transistor 62 alone provides the “AND” function. When the second transistor 62 is turned off (during X-ray exposure in the first mode), the gate of the first transistor 60 forms a floating node. This increases the source-drain capacitance of the first transistor 60 when compared with the arrangement of FIG. 3, in which the transistors 52, 54 are actively turned off. This increase in the source-drain capacitance improves the sensitivity of the pixel for the dose sensing operation.

FIG. 5 shows the read out sequence for the pixel configurations of FIGS. 3 and 4. In order to read out all pixels in a sub-array in turn, each sub-array of pixels is addressed in a similar manner to conventional read out. Thus, a row address pulse is applied to each row 44 in turn, and within the duration of each row address pulse 70, a column address pulse 72 is applied to each column 46 in turn. For the embodiment of FIG. 4, the gate of the second transistor 62 is connected to the longer row address signal, and the source of the first transistor 60 is connected to the shorter column address pulse. This ensures the first transistor 60 is properly switched off.

The invention can be realised in several different technologies, all of which are of interest in medical image sensors. FIGS. 6 to 9 show cross-sections of the main technologies of interest for medical image sensors. The specific layers in these cross sections will not be described in detail, as the implementation of the invention will be routine to those skilled in the art. In particular, the invention involves only a change in the layout the components of each pixel, particularly the TFTs, and these changes do not require any change to the existing processing technologies. FIGS. 6 to 9 are provided simply for illustrating some of the different possible implementations of the invention.

FIG. 6 shows a planar TFT-diode configuration, in which the TFTs (only one 80 shown in FIG. 6) are arranged laterally with respect to the photodiode structure 82. FIG. 6 shows the gate line 84, the read out line 86 and the common electrode 88.

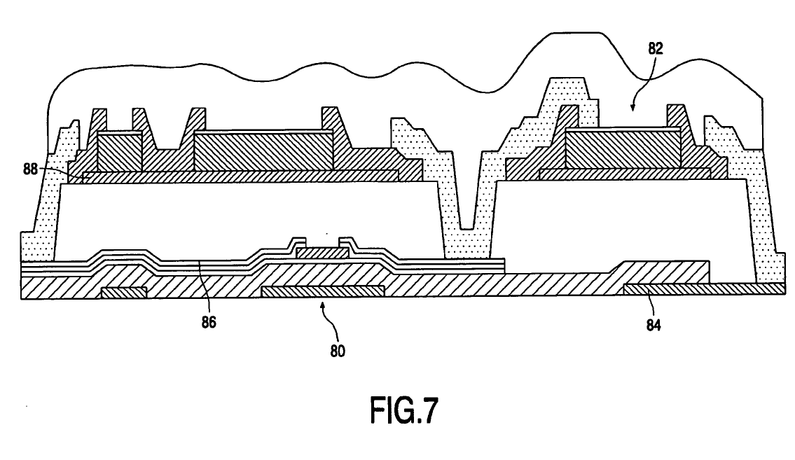

FIG. 7 shows a multi-level ‘diode on top’ technology, in which the photodiode structure 82 is provided above the TFTs (only one 80 again shown in FIG. 7). FIG. 7 also shows the gate line 84, the read out line 86 and the common electrode 88.

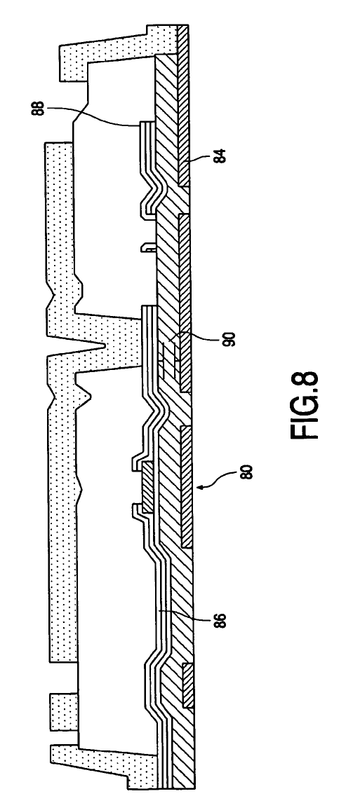

FIG. 8 shows an ‘electrode on top’ technology, suitable for direct conversion X-ray detectors. The direct conversion element requires a capacitor 90, which is provided laterally of the TFTs (only one 80 again shown in FIG. 8). FIG. 8 also shows the gate line 84, the read out line 86 and the common electrode 88.

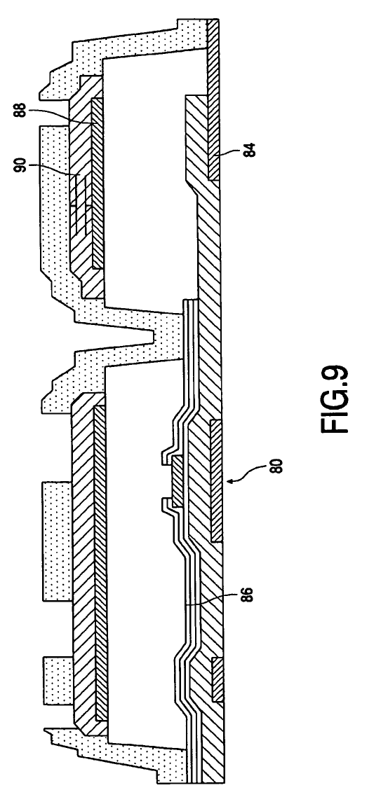

FIG. 9 shows multi-level ‘capacitor on top’ technology, suitable for direct conversion detectors. The direct conversion element again requires a capacitor 90, which is provided above the TFTs (only one 80 again shown in FIG. 9). FIG. 8 also shows the gate line 84, the read out line 86 and the common electrode 88.

FIGS. 10 to 12 show in more detail how the pixel layout of FIG. 4 (by way of example) may be implemented using different technologies. The same reference numerals are used in these Figures to denote the same components, and description is not repeated.

FIG. 10 shows a pixel design for the planar TFT-diode technology. The photodiode is defined between a pixel electrode 100 and the underlying common electrode 102. The row control line 44, column control line 46 and read out line 42 are shown, as well as the two TFTs 60,62. An internal sub-array line 104 provides connection of the read out line 42 between different pixels within each row of pixels within the sub-array. Of course, the space occupied by the two TFTs 60,62 reduces the area of the pixel electrode 100 (photodiode).

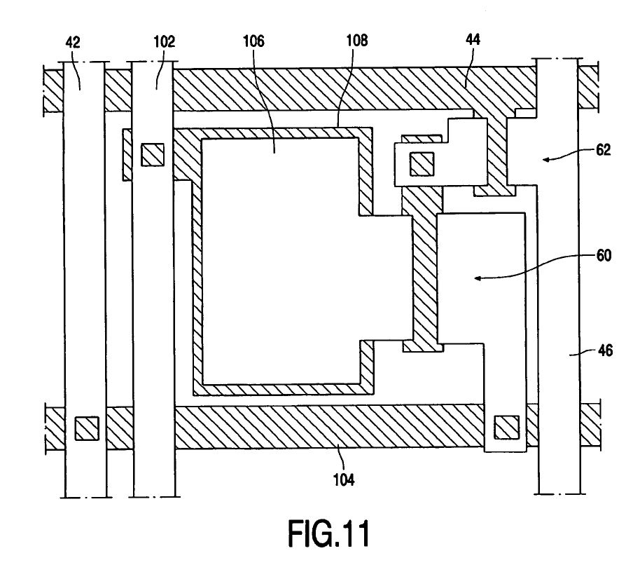

FIG. 11 shows a pixel design for electrode on top technology, where a storage capacitor 106 is made between the gate metal layer (defining the lower electrode 108) and source-drain metal of the TFTs 60,62. Each pixel in a column is connected to the common electrode by additional column conductors 102, which may themselves be connected together outside the pixel area.

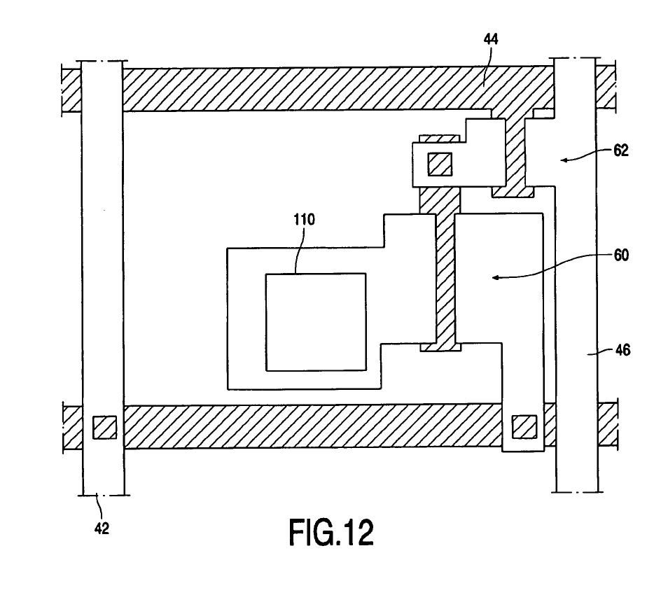

FIG. 12 shows a pixel design for ‘on top’ technologies, i.e. a design suitable for both ‘diode on top’ and ‘electrode on top’ technologies. In this case, the pixel electrode has a contact area 110 above which is defined the photodiode or direct conversion device.

As described above, the device of the invention is capable of integrated dose sensing, by using the intrinsic TFT source-drain capacitance of the read-out TFT as a tapping capacitance. The source-drain capacitance of the read-out TFT is increased when the gate electrode is a floating node, compared to the intrinsic source-drain capacitance, as employed in the pixel layout of FIG. 4. This means the dose-sensing signal will be increased. An additional approach in order to exactly match the dose sensing signal to the read-out signal, is to add additional capacitance to the floating gate node (the gate of transistor 60) of the circuit of FIG. 4. This reduces the intrinsic source-drain capacitance of the read-out TFT, without unduly increasing the charging requirements of the control TFT. The ideal value of the nodal capacitance can be determined by detailed simulation and modelling.

This additional capacitor enables the dose sensing signal to be matched to the read-out signal. In an ideal design, the stray TFT capacitance used to generate the dose sensing signal would be equal to the pixel capacitance divided by the number of pixels in the sub-array. This means that the charge sensitive amplifier would not have to undergo range changing on transition from the dose sensing to the pixel read out function.

In fact, the stray capacitance is much larger than the optimum value. The pixel capacitance may about 2 pF and there may be about 1000 pixels in the sub array, making a target value of 2 fF per pixel.

With the read out TFT 60 (FIG. 4) is in the off state, its gate is floating so that the stray capacitance consists of the source-drain capacitance (˜2 fF) in parallel with the source-gate capacitance (25 fF) and gate-drain capacitance (25 fF), the latter two being in series giving a total of about 12 fF.

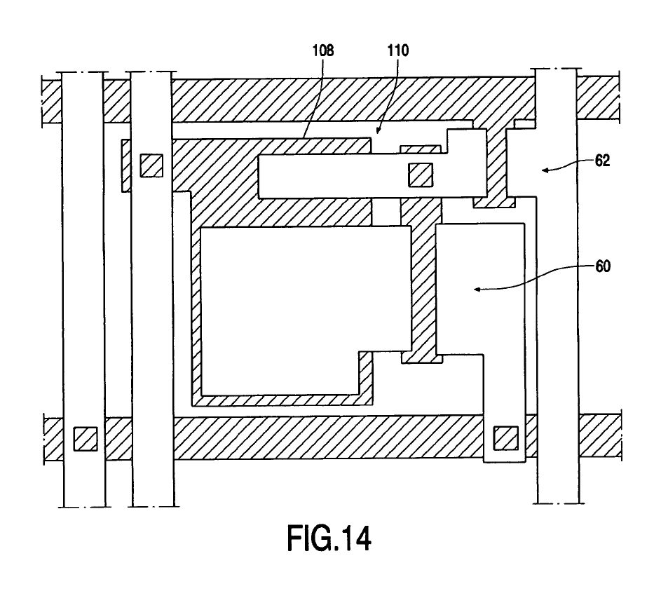

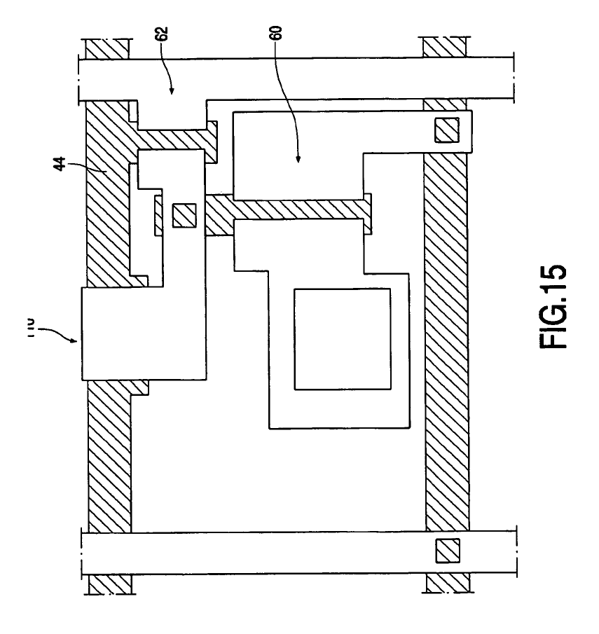

The additional capacitor, for charge sharing, can be positioned either between the conversion element and the common electrode (as shown in FIGS. 13 and 14 below) or between the conversion element and the gate of the control TFT 62 (as shown in FIG. 15 below).

FIGS. 13 to 15 correspond to FIGS. 10 to 12, but additionally show the positioning of this nodal capacitance, for each technology.

FIG. 13 corresponds to FIG. 10, and shows the additional node capacitor 110 between the gate of the first TFT 60 (read-out TFT) and the common line 102. FIG. 14 corresponds to FIG. 11, and again shows the additional node capacitor 110 between the gate of the first TFT 60 (read out TFT) and the electrode 108.

FIG. 15 corresponds to FIG. 12, and shows the nodal capacitance between the gate of the first transistor 60 (the read-out TFT) and gate of the second TFT 62. In particular, the capacitor 110 is defined between the gate of the first TFT 60 and the row control signal line 44. Some coupling of the control gate signal into the read-out line will be experienced, but this does not affect the read-out operation, provided the read-out amplifiers are not overloaded.

During the dose sensing operation, a processing unit collects the dose signals from each read out amplifier. It may be arranged to sum the dose signals of selected sub-arrays, and provide these as a first dose output. Furthermore, a dose rate signal may also be derived from the selected dose sensing sub-arrays, to indicate the dose per unit time.

As explained above, the exposure control is preferably carried out to provide the best image contrast for an area of the image of particular interest. Therefore, it is possible for a processing unit to analyse a particular pattern of sub-arrays of interest for the particular X-ray examination taking place.

Furthermore, different weights can be assigned to certain dose sensing pixel sub-arrays to obtain a weighted dose signal and dose rate signal.

The dose sensing signals can be analysed in the analogue domain or after sampling to obtain exposure information. When a given condition has been reached, analysis of the sampled outputs results in termination of the X-ray exposure period which is followed by the read out stage. The X-ray exposure may be pulsed, and the exposure control then dictates when the X-ray exposure ceases.

In the examples described above, the dose sensing pixels are shown schematically, in each case, as forming a block of 4×4 pixels. Of course, this is not necessarily the case, and in fact the dose sensing pixels will be grouped in much larger groups. Of course, the array will not necessarily have the same number of rows and columns, and indeed the pixel blocks which share a common dose sensing signal output will not necessarily be square.

The manufacturing processes involved in forming the solid state device have not been described in detail. The pixel configuration of the invention can be achieved using the thin film techniques applied for conventional cells. Typically, such devices are amorphous or polycrystalline silicon devices fabricated using thin film techniques.

Various modifications will be apparent to those skilled in the art.

1. An X-ray detector apparatus comprising an array of detector pixels (20), each pixel comprising a conversion element (26;260) for converting incident radiation into a charge flow, a charge storage element (28) and a switching arrangement (50) enabling the charge stored to be provided to an output of the pixel, wherein the array of pixels is arranged into a plurality of sub-arrays (40), each sub array comprising a plurality of pixels, the pixels in each sub-array (40) sharing a common output (42), and wherein the detector apparatus is operable in two modes, a first mode in which the switching arrangement (50) is turned off and charge flow in response to incident radiation is partially coupled through an off-capacitance of the switching arrangement to the output (42) for measurement as a dose sensing signal, and a second mode in which the switching arrangement (50) is turned on to allow charge to flow between the charge storage element and the output for measurement as a detection signal, and wherein the switching arrangement is turned on by first and second control signals which enable a single pixel within the sub-array to be selected.

2. Apparatus as claimed in claim 1, further comprising a conversion layer for converting an incident X-ray signal into an optical signal, and wherein the conversion element comprises an optical sensor.

3. Apparatus as claimed in claim 2, wherein optical sensor comprises a photodiode (26).

4. Apparatus as claimed in claim 3, wherein the charge storage element comprises the self-capacitance of the photodiode (26).

5. Apparatus as claimed in claim 1, wherein the conversion element comprises a photoconductor (260).

6. Apparatus as claimed in any preceding claim, wherein the switching arrangement comprises first and second thin film transistors (52,54) in series between the conversion element (26;260) and the output (42), one of the transistors (54) being gated by a row select control signal and the other of the transistors (52) being gated by a column select control signal.

7. Apparatus as claimed in any one of claims 1 to 5, wherein the switching arrangement comprises a first thin film transistor (60) in series between the conversion element (26;260) and the output (42) and a second thin film transistor (62), wherein the second thin film transistor (62) is gated by a first control signal for switching a second control signal to the gate of the first transistor (60).

8. Apparatus as claimed in claim 7, wherein each pixel further comprises an additional capacitor between the gate of the first transistor (60) and the conversion element (26).

9. Apparatus as claimed in any preceding claim, wherein the pixels are arranged in rows and columns, wherein each sub-array (40) comprises a plurality of rows and columns.

10. Apparatus as claimed in claim 9, comprising a plurality of first control lines (44) for carrying the first control signals, the number of first control lines corresponding to the number of rows in each sub-array (40) with each first control line (44) being provided to one row of each sub-array (40), and a plurality of second control lines (46) for carrying the second control signals, the number of second control lines (46) corresponding to the number of columns in each sub-array (40) with each second control line (46) being provided to one column of each sub-array (40).

11. Apparatus as claimed in claim 9 or 10, wherein a read out amplifier (36) is provided for each sub-array of pixels.

12. An X-ray examination apparatus comprising:
an X-ray source (10) for exposing an object to be examined to X-ray energy; and
an X-ray detector (14) as claimed in any preceding claim, for receiving an X-ray image after attenuation by the object to be examined.
